(19) United States
(12) Patent Application Publication
(10) Pub. No.: US 2026/0000561 A1
(43) Pub. Date: Jan. 1, 2026

(54) MOBILE EXAMINATION TABLE WITH RETRACTABLE WHEELS

(71) Applicant: The Brewer Company, LLC, Menomonee Falls, WI (US)

(72) Inventors: Curtis LEPPANEN, Thiensville, WI (US); Nick MARTIN, Milwaukee, WI (US); Aaron FREUND, Sussex, WI (US); Jack DEBRAAL, Oconomowoc, WI (US); Mike HOFT, Germantown, WI (US)

(73) Assignee: The Brewer Company, LLC, Menomonee Falls, WI (US)

(21) Appl. No.: 19/252,921

(22) Filed: Jun. 27, 2025

Related U.S. Application Data
(60) Provisional application No. 63/666,157, filed on Jun. 29, 2024.

(51) Int. Cl. A61G 13/10; A61G 13/00; A61G 13/08; A61G 13/12

(52) U.S. Cl. CPC A61G 13/104; A61G 13/0018; A61G 13/08; A61G 13/107; A61G 13/1225; A61G 13/123

(57) ABSTRACT

A method of moving an examination table including activating an actuator from a user interface located in front of an examination table to move a pair of retractable wheels from a retracted position to an extended position; and moving the examination table on the pair of retractable wheels and a pair of front wheels by exerting a force on a front portion of the examination table.

### CROSS-REFERENCE TO RELATED PATENT APPLICATIONS

This Application claims the benefit of U.S. Provisional Application No. 63/666,157 filed on Jun. 29, 2024 entitled MOBILE EXAMINATION TABLE WITH RETRACTABLE WHEELS which is incorporated herein in its entirety.

### BACKGROUND

The present application relates to medical examination tables and, more specifically, to medical examination tables that may be moved by a user.

### SUMMARY

In some aspects, the techniques described herein relate to an examination table, including: a base having a front end and a rear end and a longitudinal axis extending between the front end and the rear end; a patient support secured to the base; a patient support surface operatively secured to the patient support; a first wheel system including at least one wheel secured to the base; and at least a pair of feet secured to the base; and a retractable wheel system having at least one retractable wheel that is retractable and extendable relative to the base and being closer to a center of gravity of the examination table than the first pair of wheels.

In some aspects, the techniques described herein relate to a method of moving an examination table including: activating an actuator from a user interface located in front of an examination table to move a pair of retractable wheels from a retracted position to an extended position; and moving the examination table on the pair of retractable wheels and a pair of front wheels by exerting a force on a front portion of the examination table.

In some aspects, the techniques described herein relate to a medical examination table including: a base having a bottom, a top, a front, and a rear; a vertical support coupled to the base between the front and the rear of the base; a patient support moveable independent of the base between a lowered position and a raised position, the patient support including a backrest and a seat; a lift mechanism coupled to the vertical support of the base and the patient support; a plate, pivotably secured to the base, the plate including at least one wheel and a mounting surface; an actuator, one end pivotally secured to the vertical support and the opposite end pivotally secured to the mounting surface of the plate whereby extension and retraction of the actuator causes the plate to pivot between a raised position and a lowered position; and the at least one wheel protruding below the bottom of the base when the plate is in the lowered position.

### BRIEF DESCRIPTION OF THE DRAWINGS

FIG. 1 is a side isometric view of an examination table with the patient support in the fully raised position with retractable wheels in the fully retracted position.

FIG. 2 is a side isometric view of the examination table of FIG. 1 with the retractable wheels in the fully extended position.

FIG. 3 is the examination table of FIG. 1 with the back rest of the patient support in the fully reclined position.

FIG. 4 is a partial isometric view of the examination table showing the retractable wheel assembly.

FIG. 5 is a close-up view of FIG. 4 taken along lines 5-5 with the retractable wheels in the extended position.

FIG. 5A is a close-up view of FIG. 4 taken along lines 5-5 with the retractable wheels in the retracted position.

FIG. 5B is a close-up view of the retractable wheels assembly taken from a different perspective in the retracted position.

FIG. 6 is a bottom isometric view of the retractable wheel assembly in the retracted position.

FIG. 7 is a bottom isometric view of the retractable wheel assembly in the extended position.

FIG. 8 is the examination table of FIG. 2 with the patient support in a lowered position.

FIG. 9 is a bottom view of the examination table of FIG. 1.

FIG. 10 is a front isometric view close-up view of a user interface of the retractable wheel system.

FIG. 11 is a front close-up view of a user interface of the retractable wheel system.

FIG. 12 is a side view of the base of the examination table of FIG. 2 with the retractable wheels in the extended position.

FIG. 12A is a side view of the base of the examination table of FIG. 1 with the retractable wheels in the retracted position.

### DETAILED DESCRIPTION OF THE EXAMPLE EMBODIMENTS

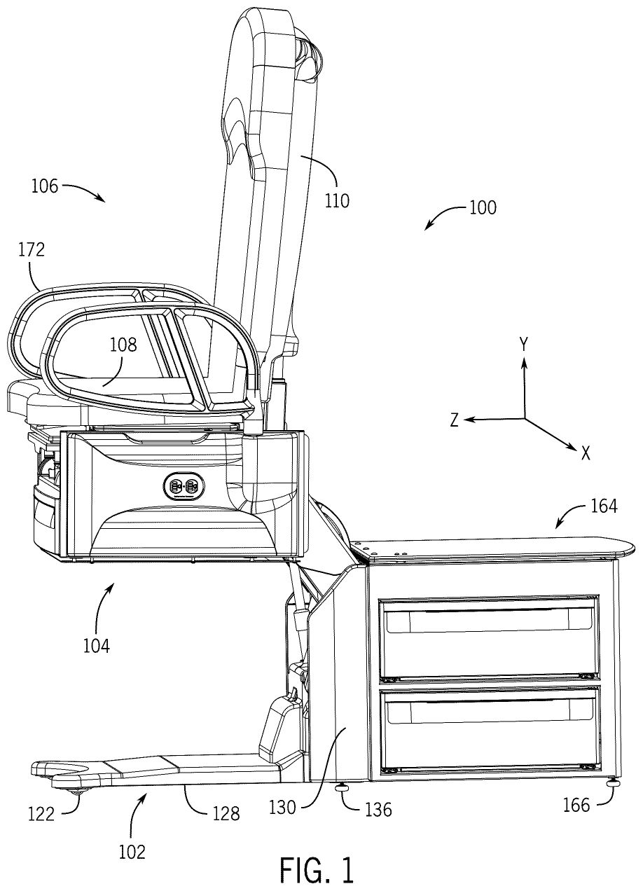

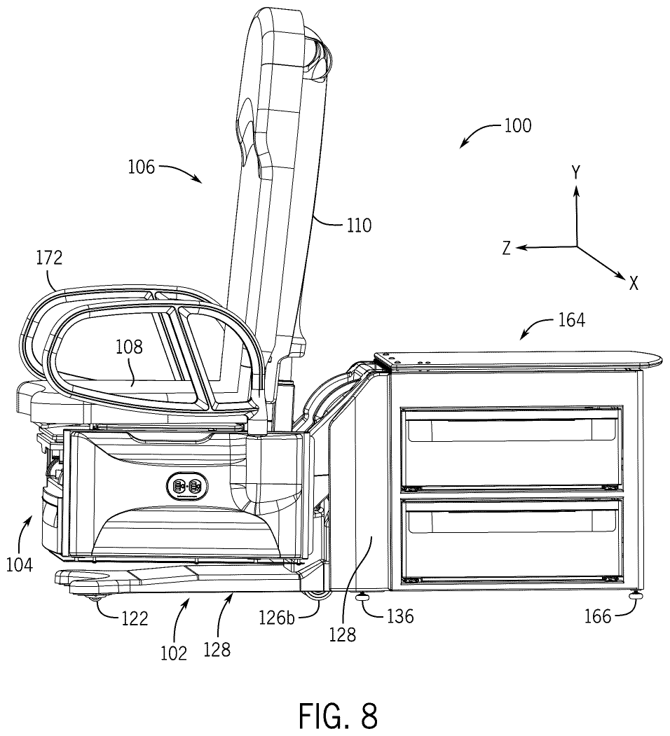

Referring to FIG. 1 and FIG. 8, an examination table 100 includes a base 102, a patient support 104 supporting a patient support surface 106 having seat portion 108 and a back rest portion 110. Patient support 104 is moved between a fully raised position and a fully lowered position relative to base 102.

Before discussing the details of examination table 100 and components thereof, it should be noted at the outset that references to “front,” “back,” “rear,” “upper,” “lower,” “right,” and “left” in this description are merely used to identify the various elements as they are oriented in the Figures, with “front,” “back,” and “rear” being relative to a patient seated in the patient support surface 106 when examination table 100 is in an in-use position. These terms are not meant to limit the element which they describe, as the various elements may be oriented differently in various applications.

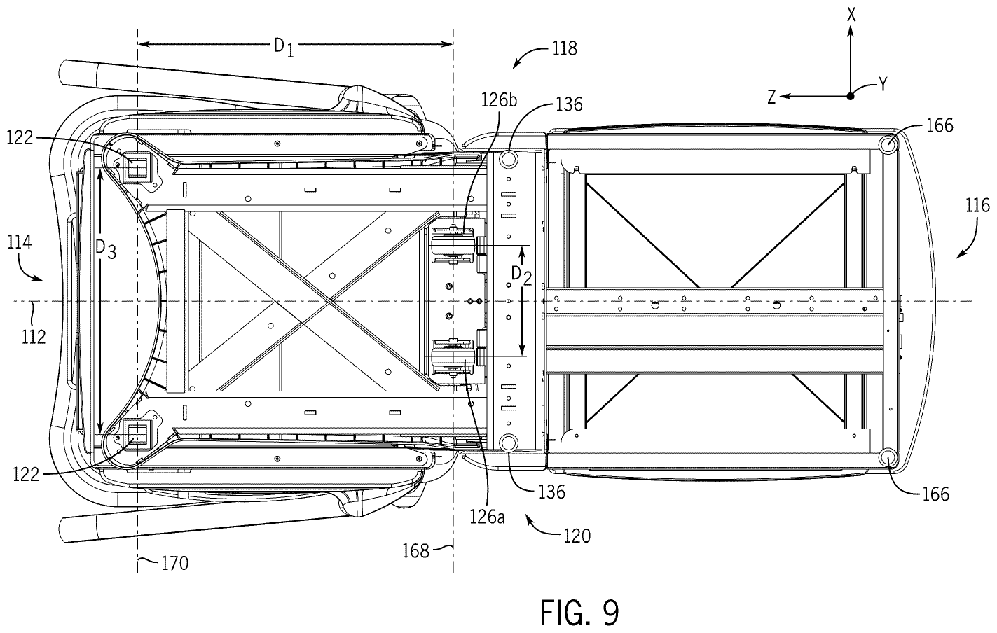

Referring to FIG. 9 a longitudinal axis 112 extends through a front end 114 and a rear end 116 of examination table 100 and is centrally located between a left side 118 and a right side 120 of examination table 100.

Examination table 100 includes a first pair of wheels 122 secured to base 102 and a retractable wheel system 124 that moves at least one wheel 126a that retracts and extends relative to a bottom 128 of base 102.

In one implementation retractable wheel system 124 includes a pair of wheels 126a and 126b. Each wheel 126a and 126b are spaced from longitudinal axis 112. Examination table 100 has a center of gravity that is located on or substantially along longitudinal axis longitudinal axis 112. As used herein the center of gravity is defined as the location in which the weight of a front portion of examination table 100 is in balance with the weight of a rear portion of the examination table 100. Where the front portion and the rear portion is the complete examination table 100. Similarly, the center of gravity between a right side of examination table 100 and the left side of examination table 100 is in balance. Stated another way, when examination table 100 is in an in-use position resting on a floor that defines a plane that is perpendicular to the direction of gravity, the weight of examination table 100 to the right of longitudinal axis 112 is the same as the weight of examination table 100 to the left of longitudinal axis 112. The center of gravity may be a point between a top of examination table 100 and a bottom of examination table 100. However the location of the center of gravity will be discussed as a projected point along the direction of gravity onto a plane defined by the front pair of wheels 122 and retractable wheel 126a when wheels 126a is in a fully extended position. Patient support 104 is movable from a lowered to a raised position by a

mechanism that changes the center of gravity of examination table 100 as patient support 104 is moved between the raised and lowered positions. The mechanism that raises and lowers support 104 is disclosed in U.S. Pat. No. 7,513,000 which is incorporated herein in its entirety. US Patent discloses the lift mechanism including a linkage system that results in movement of patient support in a way that changes the center of gravity along longitudinal axis 112 as patient support 104 is moved between a raised and lowered position.

A vertical support 130 extending upward from base 102 houses the patient table lift mechanism as described in U.S. Pat. No. 7,513,000. In one implementation the first pair of wheels 122 are secured to a front portion of base 102 and retractable wheels 126a, 126b are secured to a plate 132 pivotably secured to base 102. Wheels 126a 126b are secured to plate 132 and rotate about a wheel axel 134 fixed to plate 132. In one implementation each wheel 126a, 126b rotate about a separate wheel axel 134. Referring to FIG. 1 a pair of adjustable level legs 136 extend from base 102 adjacent vertical support 130. In one implementation retractable wheels 126a, 126b are closer to the center of gravity of examination table 100 than the first pair of wheels 122.

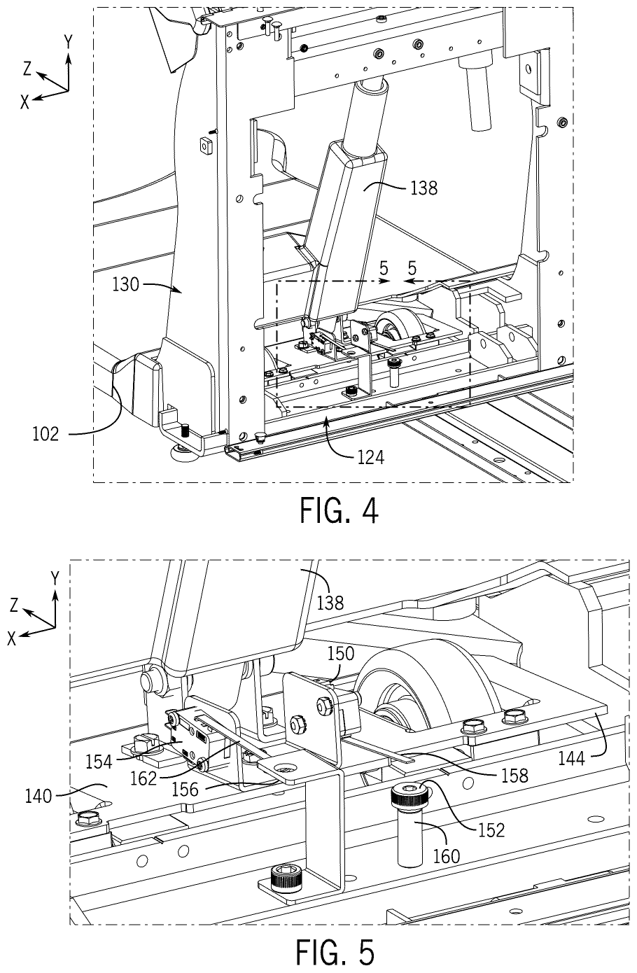

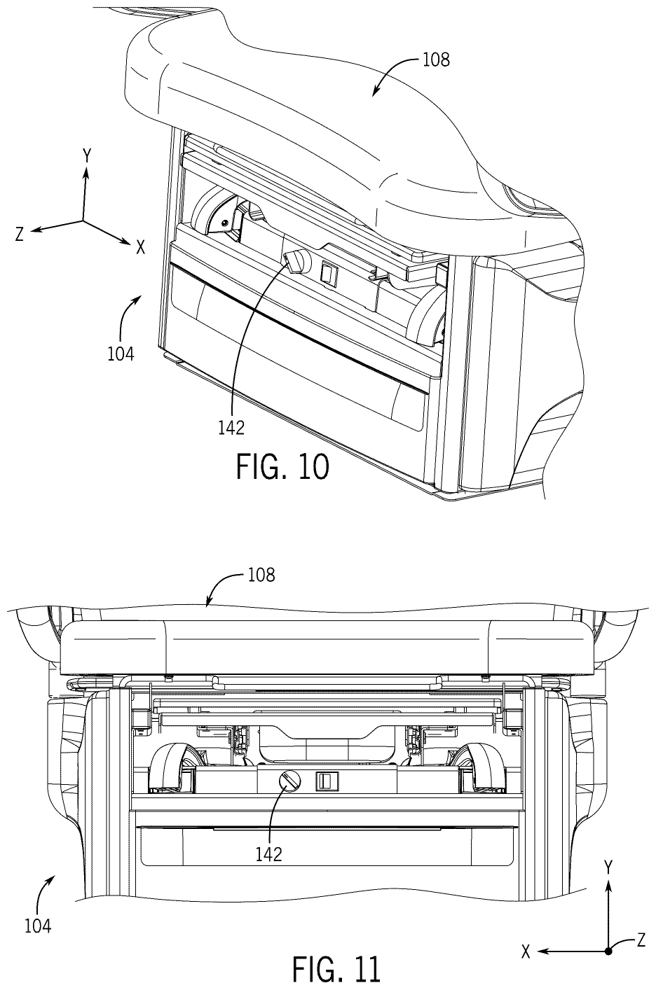

Referring to FIG. 4,-FIG. 7, retractable wheel system 124 include an actuator 138 operatively connected to a top surface 140 of plate 132 and operatively connected to region of vertical support 130 distal plate 132. Referring to FIG. 10 and FIG. 11, actuator 138 is actuated from a signal by a user interface 142 located on the front of patient support 104. In one implementation user interface 142 is located in a position on examination table other than the front of patient support 104. Further user interface 142 may be located on a structure other than patient support 104. In one implementation user interface 142 may be physically separate from examination table 100 and wirelessly connected to retractable wheel system 124 such as a separate remote control or a phone app. In one implementation actuator 138 is an electric actuator as is known in the art. However, other types of actuators known in the art may be used including but not limited to pneumatic and hydraulic actuators. It is also contemplated that retractable wheel system 124 can be a manual or mechanical system. In one implementation the manual activation of the mechanical system may be a lever or foot activated member such as a pedal or plate or other known mechanical devices known in the art and located adjacent the front end of base 102. In one implementation the manual activation device may be located on a right side, left side or rear of the base 102. In one implementation, retractable wheel system does not include a wheel brake. In one implementation, wheel system 124 includes locking cam to maintain plate 132 in the fully extended position.

Plate 132 includes a rear edge 144 and an opposing front edge 146. Plate 132 is pivotally secured to base 102 with a first hinge 148a. In one implementation a second hinge 148b is employed to pivotally secure plate 132 to base 102. However, it is contemplated that one or more hinges may be used to pivotally secure plate 132 to base 102. Actuator 138 includes a first switch 150 stops movement of actuator 138 when plate 132 is in a fully retracted position by contact with a first limit surface 152 affixed to base 102. Similarly, actuator 138 includes a second switch 154 that stops movement of plate 132 when plate 132 is in a fully extended position by contact with a second limit bracket surface 156. It is contemplated that surface 156 may be on a structure other than a bracket as is known in the art. The use of a bracket is used herein as a non-limiting example. In one implementation limit switches are incorporated into the actuator 138 as is known in the art.

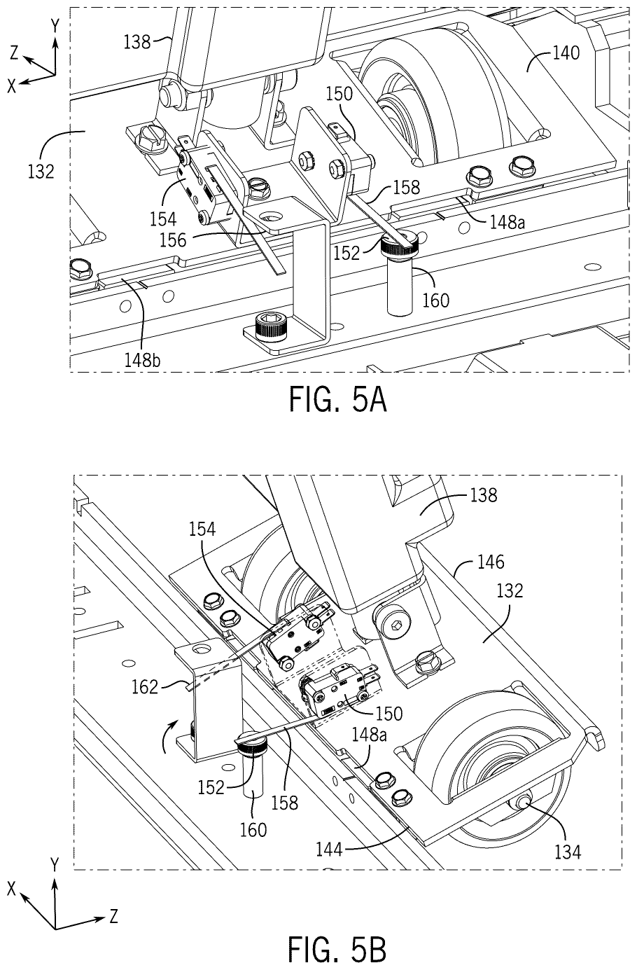

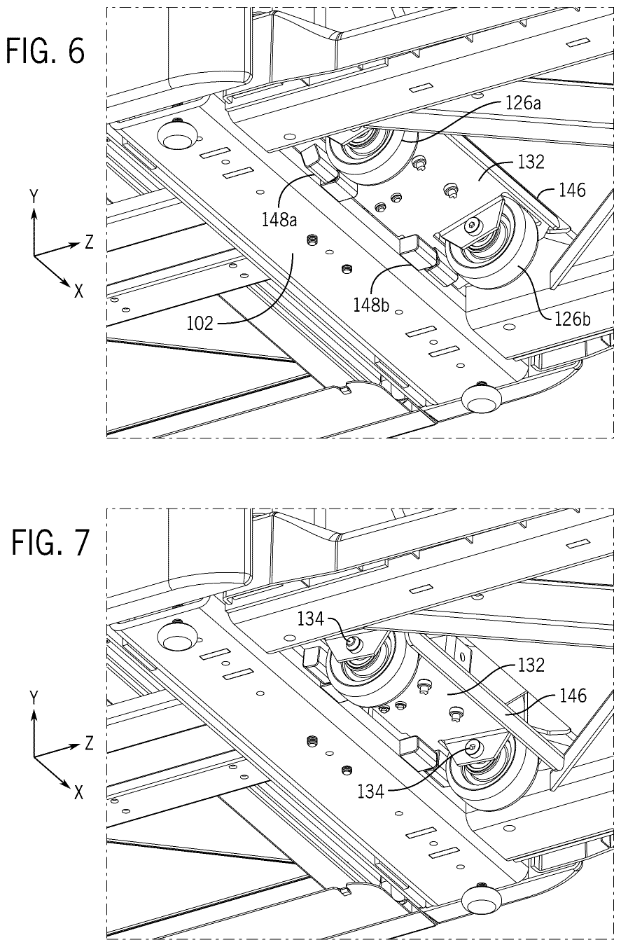

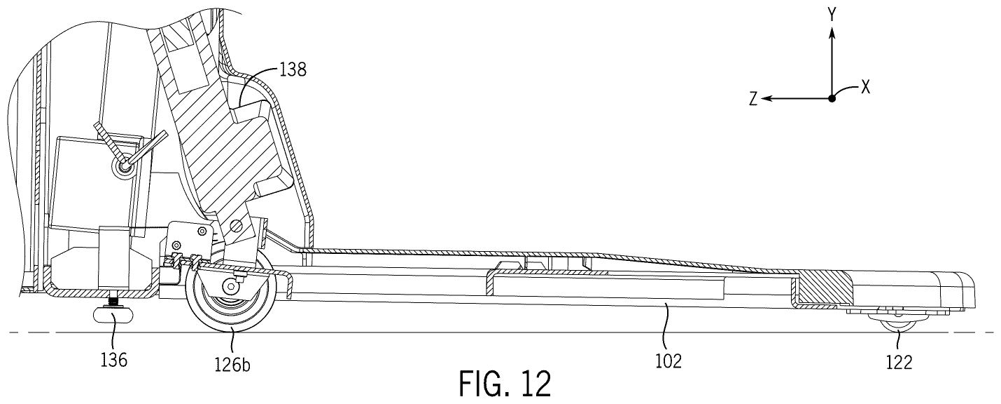

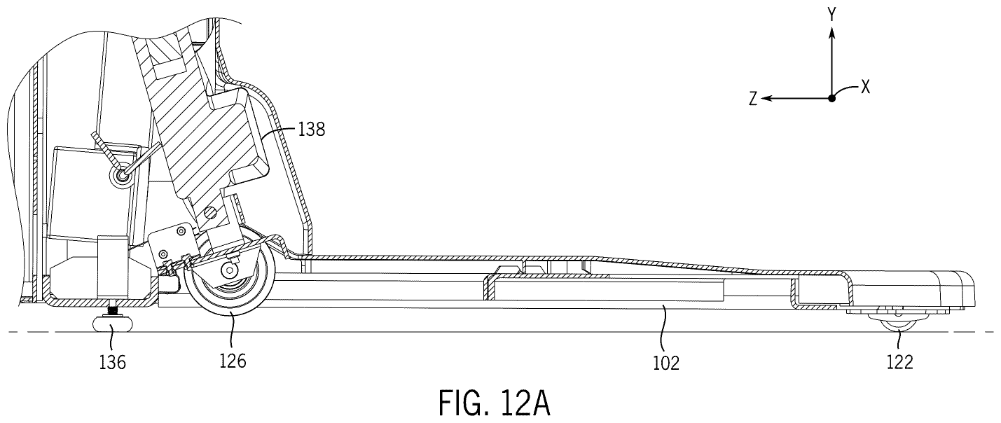

Referring to FIG. 1, FIG. 5A, FIG. 5B and FIG. 6, retractable wheel system 124 is in a retracted position. In the retracted position examination table 100 is supported by the front pair of wheels 122 and legs 136. Referring to FIG. 12 in the fully retracted position examination table 100 is in an in-use position and does not roll from the location upon which wheels 122 and adjustable level legs 136 are resting on a surface such as a medical exam room floor. In the fully retracted position, first switch 150 contacts first limit surface 152 prohibiting actuator 138 from moving plate 132 to a further retracted position. First switch 150 contacts first limit surface 152 with an arm 158. In one implementation first limit surface 152 is a top surface secured to a post 160 that is secured to base 102. In one implementation first switch 150 and/or the position of 152 is adjustable for a user to adjust the position of plate 132, wheels 126a and wheels 126b in the fully retracted position.

When a user desires to move plate 132 from the fully retracted position to a fully extended position, the user activates actuator 138 via by user interface 142 such as an electrical switch located on the front of patient support 104. By activating user interface 142 from a first state to a second state, actuator 138 is activated moving plate 132 from the retracted position to the fully extended position. User interface 142 may be a toggle switch, touch screen, rotary switch or any other type of switches known in the art. Referring to FIG. 5 and FIG. 7 plate 132 pivots about pins within first hinge 148a and second hinge 148b until a second arm 162 of second switch 154 contacts second limit bracket surface 156 thereby stopping movement of actuator 138. In one implementation a light on user interface 142 becomes illuminated upon movement of plate 132 from the fully retracted position alerting a user that examination table 100 is no longer in an in-use fixed position relative to the support surface such as a floor of an examination room. In one implementation second switch 154 and/or the position of second limit bracket surface 156 is adjustable for adjustment of position of plate 132, wheels 126a and wheels 126b in the fully extended position. In one implementation a light on user interface 142 becomes illuminated when retractable wheel system 124 is in the retracted position indicating that examination table 100 is a fixed and non-mobile orientation.

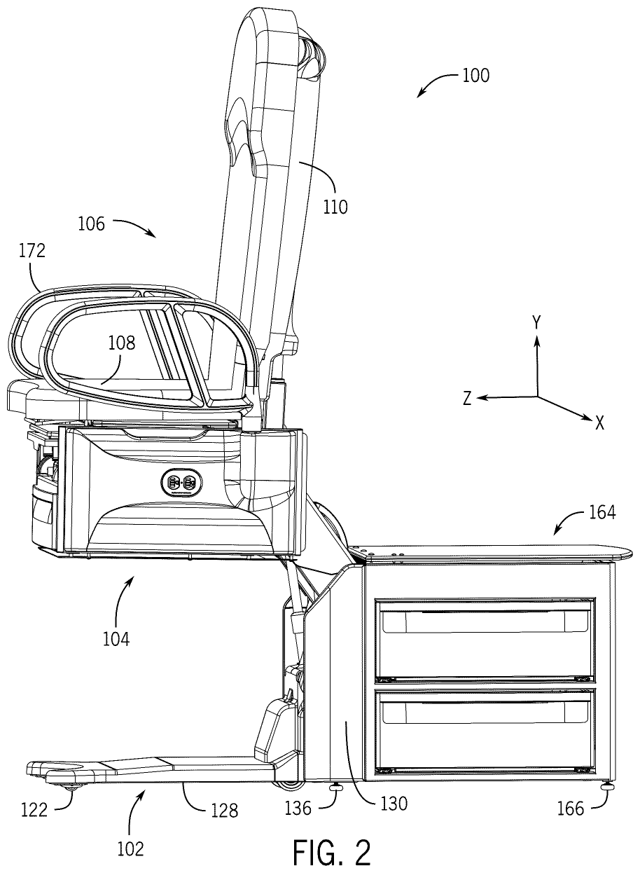

Referring to FIG. 1 and FIG. 12, in the fully retracted position a bottom portion of wheels 122 and the bottom portion of adjustable level legs 136 define a rest plane upon which examination table 100 rests in the in-use fixed position relative to the examination room. Referring to FIG. 2 and FIG. 12A as actuator 138 moves plate 132 from the fully retracted position to the fully extended position, wheels 126a and wheels 126b extend below the rest plane thereby until examination table 100 is now resting on wheels 122 and retractable wheel 126a and retractable wheel 126b. In this manner wheels 126a and wheels 126b extend below the rest plane defined by wheels 122 and adjustable level legs 136. In the fully extended position examination table 100 may be moved by a user with little effort within the strength of a wide range of medical care workers.

In one implementation examination table 100 includes a cabinet 164 that is operatively secured to base 102. Cabinet 164 includes a number of storage areas such as drawers for the storage of medical supplies used during a medical procedure. The operation of cabinet 164 is described in U.S. Pat. No. 7,513,000 identified hereinabove.

Cabinet 164 includes a pair of level support feet 166 located at the rear portion of cabinet 164. Support feet 166 contact the supporting surface of examination table 100 such as the floor of an examination room. In one implementation wheels 122, wheels 126 and 166 all sit in the rest plane when the floor of the examination room is level. In the fully extended position examination table 100 is supported by wheels 122 and retractable wheels 126a, 126b. In the fully extended position of wheels 126a and wheels 126b, level feet 136 and level feet 166 are positioned above the supporting surface such as the floor of the examination room.

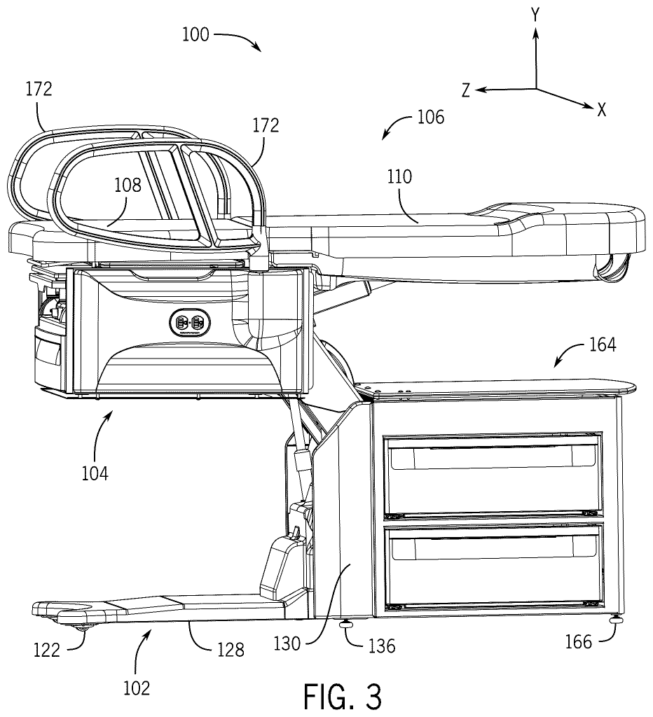

As discussed herein the center of gravity of examination table 100 will vary depending on the weight and location of the items within the cabinet storage areas as well as the position of patient support 104 and the position of back rest portion 110 of patient support surface 106. Since movement of patient support 104 between a lowered and raised position shifts the weight forward and aft along longitudinal axis 112 the center of gravity of examination table 100 will also shift. Referring to FIG. 1 and FIG. 3, back rest portion 110 can be moved between an upright position in which back rest portion 110 supports a patient's back to a lowered position in which a patient may be in a horizontal or position in which the patient is lying down on the patient back or on the patient's stomach in prone position. The center of gravity of examination table 100 will shift as back rest portion 110 is moved between a fully upright position and a fully reclined position. While it is possible to move examination table 100 with a patient located on patient support surface 106 it is preferable to move examination table 100 without a patient on patient support surface 106.

Referring to FIG. 9, in one implementation examination table 100 includes base 102, vertical support 130, patient support 104 and cabinet 164. The center of gravity in this configuration is closer to a retractable wheel axis 168 defined by wheel axels 134 of retractable wheels 126a and wheels 126b than a front wheel axis 170 defined by the first pair of wheels 122. Wherein the retractable wheel axis 168 and the front wheel axis 170 are perpendicular to longitudinal axis 112 of examination table 100. The distance between retractable wheel axis 168 and front wheel axis 170 along longitudinal axis 112 is a predetermined distance D1. In one implementation when the cabinet is empty of medical or other supplies and when patient support 104 drawers or shelves are also empty the center of gravity of examination table 100 is closer to retractable wheel axis 168 than front wheel axis 170. Additionally, in one implementation when the cabinet includes medical or other supplies and when patient support 104 includes medical or other supplies he center of gravity of examination table 100 is closer to retractable wheel axis 168 than front wheel axis 170. The center of gravity of examination table 100 remains closer to retractable wheel axis 168 than front wheel axis 170 for all positions of patient support 104 and back rest portion 110. In one implementation examination table 100 does not include a cabinet 164. The description of the location of the center of gravity discussed herein applies to both embodiments in which there is a cabinet 164 and when examination table 100 does not include a cabinet. Though the center of gravity will move distally toward the rear of the examination table 100 with the additional weight of the cabinet. In one implementation vertical support 130 includes a decorative shroud on the outer exposed portions. Vertical support 130 includes a right and left vertical support structure and an upper horizontal member extending between the right and left vertical support structures. In one implementation cabinet 164 is secured to base 102 directly and/or to vertical support 130. In one implementation examination table 100 includes level feet 136 and level feet 166. However, it is contemplated that in the embodiment in which cabinet 164 is part of examination table 100 only leveling feet 166 are included.

In one implementation the center of gravity of examination table 100 is within 25% of the distance of D1 as measured from retractable wheel axis 168. By example if the predetermined distance D1 is 20 inches, the center of gravity will be within 5 inches of retractable wheel axis 168. In one implementation the center of gravity of examination table 100 is within 10% of the distance of D1 as measured from the retractable wheel axis 168. In the non-limiting example provide above, when D1 is 20 inches the center of gravity will be within two inches of retractable wheel axis 168. In one implementation the center of gravity of examination table 100 is within 5% of the distance of D1 as measured from the retractable wheel axis 168. In the non-limiting example provide above, when D1 is 20 inches the center of gravity will be within one inch of retractable wheel axis 168. In one implementation the center of gravity is located between retractable wheel axis 168 and front wheel axis 170. In one implantation by design examination table 100 with a cabinet without any supplies in the drawers or shelves has a center of gravity at the intersection of retractable wheel axis 168 and longitudinal axis 112. In this orientation, patient support 104 is in a position between a fully raised and fully lowered orientation and back rest portion 110 is a partially reclined position between a fully upright position and a fully reclined position.

In one implementation the center of gravity is located along longitudinal axis 112. However, since cabinet 164 and patient support 104 may include medical supplies that have a weight that is not distributed equally about a left and right side of longitudinal axis 112, the center of gravity of examination table 100 may be offset from longitudinal axis 112. However, the center of gravity relative to longitudinal axis 112 will always be located between a first longitudinal axis extending through wheel 126a and a second longitudinal axis extending through wheel 126b where the first longitudinal axis and the second longitudinal axis are parallel to one another and to longitudinal axis 112 and reside in the plane defined by wheels 126a, 126b and wheels 122.

In one implementation the distance between wheels 126a and wheels 126b is a Distance D2 which is less than a distance D3 between wheels 122. In one implementation the center of gravity is positioned within 25% of longitudinal axis 112 of the distance D2. By way of example if the distance D2 between wheels 126a and 126b is 8.0 inches, the center of gravity will be located within 2.0 inches of longitudinal axis 112 in a direction that is parallel to retractable wheel axis 168. In one implementation the Distance D2 is equal to or less than D3. In one implementation the distance D2 is than 50% of D3.

The location of the center of gravity between retractable wheel axis 168 and front wheel axis 170 will result in examination table 100 being supported solely by wheels 126a, 126b and front pair of wheels 122 without any force applied to the examination table 100 by a user when the retractable wheels are in the fully extended position. In this implementation adjustable level legs 136 on base 102 and level legs 166 of cabinet 164 are not contacting the support surface when the retractable wheels are in the fully extended position,

In one implementation the center of gravity is located distal to retractable wheel axis 168. Stated another way retractable wheel axis 168 is positioned between the center of gravity and front wheel axis 170. In the event that the center of gravity is distal or aft of retractable wheel axis 168 a user will be required to assert a downward force on the front of examination table 100 to ensure that examination table 100 is supported solely by wheels 126a, 126b and wheels 122. However, the downward force required by a user is within a non-exertion effort by a wide range of medical personnel.

In one implementation retractable wheels 126a, 126b are a fixed via wheel axel 134 to plate 132 in which the wheel axel maintains in a fixed orientation with respect to base 102 as examination table 100 is moved from a stationary in-use position to a second location within the examination room. In one implementation wheels 126a, 126b are swivel caster wheels in which 126a, 126b are secured to a yoke that can pivot about a vertical axis of a swivel plate. In one implementation retractable wheel system 124 includes a single wheel such that when the single wheel is extended examination table 100 rests on only three wheels, the single retractable wheel and a pair of front wheels 122. In one implementation the retractable wheel is a roller ball wheel in which the wheel is supported in a housing that allows the roller ball to rotate in any direction. In one implementation, the retractable wheel is located in the front of base 102 while the fixed wheels are located distal the front of base 102. In this implementation the center of gravity is closer to the wheels distal the front of base 102 and not closer to the retractable wheel. In one implementation neither wheels 122 nor wheels 126a, 126b are swivel caster wheels. The geometry of the retractable wheels 126a, 126b being closer to longitudinal axis 112 than wheels 122 allows examination table 100 to be moved in a non-linear manner without the need for swivel caster wheels. In one implementation wheels 122 are non-retractable wheels that maintain a fixed orientation relative to base 102. Non-swivel caster wheels provide a functional advantage of not requiring the swivel caster wheels to be properly orientated for easy initial movement of the examination system. The fixed nature of wheels 122 and wheels 126a. 126b combined with the positioning of the wheels relative to one another and longitudinal axis 112 provides for an easier initial movement of the examination table while still permitting non-linear movement of the examination table. Referring to FIG. 12 and FIG. 12A front edge 146 of plate 132 extends downward when retractable wheel system 124 is in the extended position and extends upward when retractable wheel system 124 is in the retracted position. In one implementation opposing front edge 146 is a free end of plate 132 that is not connected to base 102 while rear edge 144 of plate 132 is distal to opposing front edge 146 and further from the front of examination table 100 than front edge 146 of plate 132.

A method of moving examination table 100 from a stationary in-use position to a mobile orientation includes activating an actuator from a user interface 142 located in front of an examination table examination table 100 such as on a front surface of patient support 104 to move a pair of retractable wheels 126a, 126b from a retracted position to an extended position; and moving the examination table 100 on the pair of retractable wheels 126a, 126b and a pair of front wheels 122 by exerting a force on a front portion of the examination table. In one implementation examination table 100 includes at least one grab bar, handle or assist handle 172 that a user may grasp to move examination table 100 from a specific location on the support surface to another location to facilitate access to the support surface under examination table 100 in the in-use position. In one implementation in which there is no grab bar and a user may move examination table 100 by asserting a force on examination table 100 in a pushing or pulling motion. In one implementation the user moves examination table 100 along a non-linear path. In one implementation wheels 126a, 126b are non-caster wheels. In one implementation user interface 142 is located proximate the front of examination table 100 such that a user standing proximate the front of examination table 100 can manipulate the user interface to activate retractable wheel system 124 between a retracted position and extended position.

### Exemplary Clauses

Clause 1. An examination table, comprising: a base having a front end and a rear end and a longitudinal axis extending between the front end and the rear end; a patient support secured to the base; a patient support surface operatively secured to the patient support; a first wheel system including at least one wheel secured to the base; and a pair of feet secured to the base; and a retractable wheel system having at least one retractable wheel that is retractable and extendable relative to the base and being closer to a center of gravity of the examination table than the first pair of wheels.

Clause 2. The examination table of clause 1, wherein the retractable wheel system includes at least a pair of wheels each wheel being located a first distance from the longitudinal axis, and each wheel of the first pair of wheels being located a second distance from the longitudinal axis that is greater than the first distance.

Clause 3. The examination table of clause 1, wherein the center of gravity is located along the longitudinal axis.

Clause 4. The examination table of clause 1, further including a cabinet housing secured to the base.

Clause 5. The examination table of clause 1, wherein the pair of retractable wheels are movable from a first retracted position within the base above a support plane defined by the pair of front wheels and the pair of feet, to a second extended position below the support plane.

Clause 6. The examination table of clause 5, further including an actuator that moves the retractable wheels between the first retracted position and the second extended position.

Clause 7. The examination table of clause 6, further including a first limit and a second limit, limiting a movement of the actuator to a first position moving the pair of retractable wheels to a fully retracted position and to a second position where the pair of retractable wheels are in a fully extended position respectively.

Clause 8. The examination table of clause 7, further including a first switch and second switch operatively contacting the first limit and the second limit in the fully retracted and the fully extended position respectively, wherein at least one of the first switch, second switch, first limit and second limit is adjustable to modify the fully retractable position and the fully extended position.

Clause 9. The examination table of clause 5, further including a plate secured to the base with a pivot, each of the pair of retractable wheels being rotatably secured to the plate.

Clause 10. The examination table of clause 6, wherein the actuator is located within the base.

Clause 11. The examination table of clause 1, further including a handle positioned proximate a front portion of the examination table, wherein an operator can move the examination table by directing the examination table through the handle.

Clause 12. The examination table of clause 11, wherein the handle includes a first handle portion and a second handle portion on opposite sides of a longitudinal axis of the examination table, and wherein a portion of the first handle portion and the second handle portion is located above a seat portion of the patient supporting surface.

Clause 13. The examination table of clause 11, wherein the examination table include a back rest portion movable between a horizontal position generally parallel with a seat portion and an upright position, at a non-parallel orientation with the seat portion, wherein the examination table is movable when the back rest is in both the horizontal position and upright position.

Clause 14. The examination table of clause 1, including a retractable wheel activation member in the front of the examination table, wherein a user can actuate the retractable wheels from a fully retracted position to a fully extended position and move the examination table on the front wheels and retractable wheels solely from in front of the examination table.

Clause 15. The examination table of clause 1, wherein the first pair of wheels are adjacent to the front of the base and wherein the first pair of feet are at a position distal from the first pair of wheels.

Clause 16. The examination table of clause 1, wherein the retractable wheels are between the first pair of wheels and the pair of feet.

Clause 17. The examination table of clause 1, wherein a distance between the retractable wheels and the first pair of wheels are a predetermined distance and the center of gravity is located 25% or less than a distance from the retractable wheels towards the first pair of wheels.

Clause 18. The examination table of clause 1, wherein a distance between the retractable wheels and the first pair of wheels are a predetermined distance and the center of gravity is located 5% or less than a distance from the retractable wheels towards the first pair of wheels.

Clause 19. The examination table of clause 1, wherein one of the first pair of wheels and the retractable wheel are proximate the front of the examination table and the other of the first pair of wheels and the retractable wheel is distal the front of the examination table.

Clause 20. The examination table of clause 1, further including a support activation system to raise and lower the patient support that is independent and does not cooperate with an activation system that retracts and extends the retractable wheel.

Clause 21. The examination table of clause 18, wherein the center of gravity moves along the longitudinal axis as the patient support moves up and down relative to the base, the center of gravity remains within 5% of the distance from the retractable wheels to the front wheels.

Clause 22. The examination table of clause 1, wherein the retractable wheel system includes a roller wheel, sphere, or a caster wheel.

Clause 23. A method of moving an examination table comprising: activating an actuator from a user interface located in front of an examination table to move a pair of retractable wheels from a retracted position to an extended position; and moving the examination table on the pair of retractable wheels and a pair of front wheels by exerting a force on a front portion of the examination table.

Clause 24. The method of moving an examination table of clause 23, wherein the examination table includes a base having a longitudinal axis extending between the front of the examination table and a rear end of the examination table, each wheel of the pair of retractable wheels being located a first distance from the longitudinal axis, and each wheel of the front wheels being located a second distance from the longitudinal axis that is greater than the first distance.

Clause 25. The method of moving an examination table of clause 24, wherein the examination table includes a cabinet housing secured to the base, wherein a center portion of the patient support is located closer to the front end of the base than a center portion of the cabinet.

Clause 26. The method of moving an examination table of clause 23, wherein the pair of retractable wheels are movable from a first retracted position within the base above a support plane defined by the pair of front wheels and the pair of feet, to a second extended position below the support plane by the pair of front wheels and the pair of feet.

Clause 27. The method of moving an examination table of clam 23, includes moving the examination table in a non-linear direction.

Clause 28. The method of moving an examination table of clause 27, wherein the pair of retractable wheels and the pair of front wheels are free of a brake and are non-caster wheels.

Clause 29. A medical examination table comprising: a base having a bottom, a top, a front, and a rear; a vertical support coupled to the base between the front and the rear of the base; a patient support moveable independent of the base between a lowered position and a raised position, the patient support including a backrest and a seat; a lift mechanism coupled to the vertical support of the base and the patient support; a plate, pivotably secured to the base, the plate including at least one wheel and a mounting surface; an actuator, one end pivotally secured to the vertical support and the opposite end pivotally secured to the mounting surface of the plate whereby extension and retraction of the actuator causes the plate to pivot between a raised position and a lowered position; and the at least one wheel protruding below the bottom of the base when the plate is in the lowered position.

Clause 30. The medical examination table of claim 29, wherein the base provides a storage area.

Clause 31. The medical examination table of claim 29, wherein an operator interface is provided proximal to the front of the patient support seat to operate the actuator.

Clause 32. The medical examination table of claim 29, wherein an operator interface provides a light when the at least one wheel has moved from the fully retracted position.

Clause 33. The medical examination table of claim 29, wherein a pair of wheels are attached to the front of the base.

Clause 34. The medical examination table of claim 29, wherein the pivot plate includes at least two wheels.

Clause 35. The medical examination table of claim 29, wherein the lift mechanism is coupled to the top of the base.

Clause 36. The medical examination table of claim 29, wherein an operator interface is lighted when a portion of the at least one wheel is moved from the fully retracted position and is unlit when a the ate least one wheel is in the fully retracted position.

Clause 37. An examination table, comprising: a base having a front end and a rear end and a longitudinal axis extending between the front end and the rear end; a patient support secured to the base; a patient support surface operatively secured to the patient support; and a retractable wheel system proximate the front of the base having at least one retractable wheel that is retractable and extendable relative to the base, a pair of non-retractable wheels operatively secured to the base distal the front of the base and being closer to a center of gravity of the examination table than the at least one retractable wheel; and a pair of feet secured to the base.

Although the present disclosure has been described with reference to example embodiments, workers skilled in the art will recognize that changes may be made in form and detail without departing from the spirit and scope of the defined subject matter. For example, although different example embodiments may have been described as including one or more features providing one or more benefits, it is contemplated that the described features may be interchanged with one another or alternatively be combined with one another in the described example embodiments or in other alternative embodiments. Because the technology of the present disclosure is relatively complex, not all changes in the technology are foreseeable. The present disclosure described is manifestly intended to be as broad as possible. For example, unless specifically otherwise noted, the definitions reciting a single particular element also encompass a plurality of such particular elements.

What is claimed is:

1. An examination table, comprising:
a base having a front end and a rear end and a longitudinal axis extending between the front end and the rear end;
a patient support secured to the base;
a patient support surface operatively secured to the patient support;
a first wheel system including at least one wheel secured to the base; and a pair of feet secured to the base; and
a retractable wheel system having at least one retractable wheel that is retractable and extendable relative to the base and being closer to a center of gravity of the examination table than the first pair of wheels.

2. The examination table of claim 1, wherein the retractable wheel system includes at least a pair of wheels each wheel being located a first distance from the longitudinal axis, and each wheel of the first pair of wheels being located a second distance from the longitudinal axis that is greater than the first distance.

3. The examination table of claim 1, wherein the center of gravity is located along the longitudinal axis.

4. The examination table of claim 1, further including a cabinet housing secured to the base.

5. The examination table of claim 1, wherein the pair of retractable wheels are movable from a first retracted position within the base above a support plane defined by the pair of front wheels and the pair of feet, to a second extended position below the support plane.

6. The examination table of claim 5, further including an actuator that moves the retractable wheels between the first retracted position and the second extended position.

7. The examination table of claim 6, further including a first limit and a second limit, limiting a movement of the actuator to a first position moving the pair of retractable wheels to a fully retracted position and to a second position where the pair of retractable wheels are in a fully extended position respectively.

8. The examination table of claim 7, further including a first switch and second switch operatively contacting the first limit and the second limit in the fully retracted and the fully extended position respectively, wherein at least one of the first switch, second switch, first limit and second limit is adjustable to modify the fully retractable position and the fully extended position.

9. The examination table of claim 5, further including a plate secured to the base with a pivot, each of the pair of retractable wheels being rotatably secured to the plate.

10. The examination table of claim 6, wherein the actuator is located within the base.

11. The examination table of claim 1, further including a handle positioned proximate a front portion of the examination table, wherein an operator can move the examination table by directing the examination table through the handle.

12. The examination table of claim 11, wherein the handle includes a first handle portion and a second handle portion on opposite sides of a longitudinal axis of the examination table, and wherein a portion of the first handle portion and the second handle portion is located above a seat portion of the patient supporting surface.

13. The examination table of claim 11, wherein the examination table include a back rest portion movable between a horizontal position generally parallel with a seat portion and an upright position, at a non-parallel orientation with the seat portion, wherein the examination table is movable when the back rest is in both the horizontal position and upright position.

14. The examination table of claim 1, including a retractable wheel activation member in the front of the examination table, wherein a user can actuate the retractable wheels from a fully retracted position to a fully extended position and move the examination table on the front wheels and retractable wheels solely from in front of the examination table.

15. The examination table of claim 1, wherein the first pair of wheels are adjacent to the front of the base and wherein the first pair of feet are at a position distal from the first pair of wheels.

16. The examination table of claim 1, wherein the retractable wheels are between the first pair of wheels and the pair of feet.

17. The examination table of claim 1, wherein a distance between the retractable wheels and the first pair of wheels are a predetermined distance and the center of gravity is located 25% or less than a distance from the retractable wheels towards the first pair of wheels.

18. The examination table of claim 1, wherein a distance between the retractable wheels and the first pair of wheels are a predetermined distance and the center of gravity is located 5% or less than a distance from the retractable wheels towards the first pair of wheels.

19. The examination table of claim 1, wherein one of the first pair of wheels and the retractable wheel are proximate the front of the examination table and the other of the first pair of wheels and the retractable wheel is distal the front of the examination table.

20. The examination table of claim 1, further including a support activation system to raise and lower the patient support that is independent and does not cooperate with an activation system that retracts and extends the retractable wheel.

21. The examination table of claim 18, wherein the center of gravity moves along the longitudinal axis as the patient support moves up and down relative to the base, the center of gravity remains within 5% of the distance from the retractable wheels to the front wheels.

22. The examination table of claim 1, wherein the retractable wheel system includes a roller wheel, sphere, fixed or a caster wheel.

23. A method of moving an examination table comprising:
activating an actuator from a user interface located in front of an examination table to move a pair of retractable wheels from a retracted position to an extended position; and
moving the examination table on the pair of retractable wheels and a pair of front wheels by exerting a force on a front portion of the examination table.

24. A medical examination table comprising:
a base having a bottom, a top, a front, and a rear;
a vertical support coupled to the base between the front and the rear of the base;
a patient support moveable independent of the base between a lowered position and a raised position, the patient support including a backrest and a seat;
a lift mechanism coupled to the vertical support of the base and the patient support;
a plate, pivotably secured to the base, the plate including at least one wheel and a mounting surface;
an actuator, one end pivotally secured to the vertical support and the opposite end pivotally secured to the mounting surface of the plate whereby extension and retraction of the actuator causes the plate to pivot between a raised position and a lowered position; and
the at least one wheel protruding below the bottom of the base when the plate is in the lowered position.
